(19) United States
(12) Patent Application Publication
(10) Pub. No.: US 2022/0109786 A1
(43) Pub. Date: Apr. 7, 2022

(54) ENDOSCOPE SYSTEM, ADAPTOR USED FOR ENDOSCOPE, AND METHOD OF OPERATING ENDOSCOPE

(71) Applicant: OLYMPUS CORPORATION, Tokyo (JP)

(72) Inventors: Akira MATSUSHITA, Tokyo (JP); Naoyuki MIYASHITA, Tokorozawa-shi (JP); Takuma DEZAWA, Tokyo (JP)

(73) Assignee: OLYMPUS CORPORATION, Tokyo (JP)

(21) Appl. No.: 17/493,373

(22) Filed: Oct. 4, 2021

(30) Foreign Application Priority Data
Oct. 7, 2020 (JP) 2020-169640

(51) Int. Cl. H04N 5/235; H04N 5/04; G02B 23/24

(52) U.S. Cl. CPC H04N 5/2354; H04N 2005/2255; G02B 23/2461; H04N 5/04

(57) ABSTRACT

An endoscope system includes an endoscope including an insertion unit and a sensor. The sensor is disposed at a distal end of the insertion unit. The sensor unit includes a motion sensor and a light source. The motion sensor is configured to execute a measurement operation and generate a measurement value indicating a measurement result. The light source is configured to generate light indicating the measurement operation at a timing that is synchronized with the measurement operation. The insertion unit includes an imaging device disposed at the distal end and configured to receive the light generated by the light source and generate an image.

### BACKGROUND OF THE INVENTION

### Field of the Invention

The present invention relates to an endoscope system, an adaptor used for an endoscope, and a method of operating an endoscope.

Priority is claimed on Japanese Patent Application No. 2020-169640, filed on Oct. 7, 2020, the content of which is incorporated herein by reference.

### Description of Related Art

Industrial endoscope devices have been used for inspection of abnormalities (damage, corrosion, and the like) inside boilers, gas turbines, automobile engines, pipes, and the like. In inspection using an endoscope, it is required to record evidence that an inspection target has been inspected. A visual field of an endoscope is narrow compared to the size of an inspection target. In addition, an inspection target has many periodic structures. Therefore, it is difficult for a user to determine an inspected portion on the basis of an image acquired by an endoscope.

A technique of reconfiguring a three-dimensional image on the basis of a two-dimensional image such as structure from motion (SfM) has been proposed. By using this technique, a three-dimensional image of an inspection target including an inspected portion is obtained.

However, in a case in which the quality of a two-dimensional image is poor or the number of two-dimensional images is not sufficient, there is a possibility that reconfiguration of a three-dimensional image fails. Therefore, a technique of disposing a sensor such as a motion sensor (inertial sensor) in the distal end of an endoscope is desired. Even if obtaining a three-dimensional image is not possible, information of an inspection position can be obtained by using a sensor. By associating a two-dimensional image and positional information with each other, the two-dimensional image and the positional information function as evidence of inspection.

An image generated by an imaging device and sensor data acquired by a sensor need to be temporally synchronized with each other. By supplying an imaging device and a sensor with a clock generated by one oscillator, the imaging device and the sensor can share the clock. In this case, a two-dimensional image and information of an inspection position are temporally synchronized with each other.

A technique disclosed in Japanese Unexamined Patent Application, First Publication No. 2019-201757 offers a capsule endoscope that generates an image including a synchronization signal. The capsule endoscope wirelessly transmits the image, and a reception device disposed outside a human body receives the image. The synchronization signal is detected from the received image. In order to synchronize an image and sensor data with each other, there is a possibility that this synchronization signal is used.

### SUMMARY OF THE INVENTION

According to a first aspect of the present invention, an endoscope system includes an endoscope including an insertion unit and a sensor. The sensor is disposed at a distal end of the insertion unit. The sensor includes a motion sensor and a light source. The motion sensor is configured to execute a measurement operation and generate a measurement value indicating a measurement result. The light source is configured to generate light indicating the measurement operation at a timing that is synchronized with the measurement operation. The insertion unit includes an imaging device disposed at the distal end and configured to receive the light generated by the light source and generate an image.

According to a second aspect of the present invention, in the first aspect, the endoscope system may further include a processor configured to detect the light generated by the light source by processing the image and associate a time point at which the measurement operation is executed and a time point at which the image is generated with each other so as to associate the measurement value and the image with each other.

According to a third aspect of the present invention, in the second aspect, the light source may be configured to generate the light at a first timing at which the motion sensor starts the measurement operation. The processor may be configured to detect the light generated at the first timing by the light source.

According to a fourth aspect of the present invention, in the third aspect, the light source may be configured to generate the light at a second timing at which the motion sensor stops the measurement operation. The processor may be configured to detect the light generated at the second timing by the light source.

According to a fifth aspect of the present invention, in the third aspect, the motion sensor may include an acceleration sensor configured to execute the measurement operation and an angular velocity sensor configured to execute the measurement operation.

According to a sixth aspect of the present invention, in the third aspect, the light source may be configured to generate light indicating that the measurement operation continues while the motion sensor executes the measurement operation. The processor may be configured to detect the light indicating that the measurement operation continues by processing the image.

According to a seventh aspect of the present invention, in the third aspect, the endoscope system may further include a memory configured to store the measurement value and the image associated with each other.

According to an eighth aspect of the present invention, in the first aspect, the light source may be capable of switching between a first state in which the light source generates light and a second state in which the light source stops light-emission. The pattern in which the first state occurs may correspond to the state of the measurement operation.

According to a ninth aspect of the present invention, in the eighth aspect, the number of periods during which the first state occurs may correspond to the state of the measurement operation.

According to a tenth aspect of the present invention, in the eighth aspect, the length of a period during which the first state continues may correspond to the state of the measurement operation.

According to an eleventh aspect of the present invention, in the first aspect, the sensor may be attachable to and detachable from the distal end.

According to a twelfth aspect of the present invention, in the eleventh aspect, the sensor may be disposed in an optical adaptor for observation using the endoscope.

According to a thirteenth aspect of the present invention, in the first aspect, the light source may be configured to generate visible light.

According to a fourteenth aspect of the present invention, in the first aspect, the light source may be configured to generate one of infrared light and ultraviolet light.

According to a fifteenth aspect of the present invention, in the first aspect, the light source may be configured to generate light having a wavelength corresponding to the state of the measurement operation.

According to a sixteenth aspect of the present invention, an adaptor used for an endoscope is connected to the distal end of an insertion unit included in the endoscope. The adaptor includes a motion sensor, a signal output circuit, and a light source. The motion sensor is configured to execute a measurement operation and generate a measurement value indicating a measurement result. The signal output circuit is configured to output a control signal indicating a timing of the measurement operation to the motion sensor. The light source is configured to generate light indicating the measurement operation at the timing indicated by the control signal.

According to a seventeenth aspect of the present invention, a method of operating an endoscope includes a measurement step, a light-emission step, and an imaging step. The measurement step causes a motion sensor disposed at a distal end of the endoscope to execute a measurement operation and generate a measurement value indicating a measurement result. The light-emission step causes a light source disposed at the distal end to generate light indicating the measurement operation at a timing that is synchronized with the measurement operation. The imaging step causes an imaging device disposed at the distal end to receive the light generated by the light source and generate an image.

### BRIEF DESCRIPTION OF THE DRAWINGS

FIG. 1 is a block diagram showing a configuration of an endoscope system according to a first embodiment of the present invention.

FIG. 2 is a block diagram showing a configuration of a motion sensor included in the endoscope system according to the first embodiment of the present invention.

FIG. 3 is a flow chart showing a procedure of an operation of the endoscope system according to the first embodiment of the present invention.

FIG. 4 is a timing chart showing an operation of the endoscope system according to the first embodiment of the present invention.

FIG. 5 is a block diagram showing a configuration of a light source included in the endoscope system according to the first embodiment of the present invention.

FIG. 6 is a block diagram showing a configuration of an endoscope system according to a second embodiment of the present invention.

FIG. 7 is a flow chart showing a procedure of an operation of the endoscope system according to the second embodiment of the present invention.

FIG. 8 is a block diagram showing a configuration of an endoscope system according to a third embodiment of the present invention.

FIG. 9 is a flow chart showing a procedure of an operation of the endoscope system according to the third embodiment of the present invention.

FIG. 10 is a block diagram showing a configuration of a distal end of an insertion unit included in an endoscope system according to a fourth embodiment of the present invention.

FIG. 11 is a block diagram showing a configuration of an optical adaptor included in the endoscope system according to the fourth embodiment of the present invention.

FIG. 12 is a block diagram showing a configuration of an endoscope system according to an embodiment of a first invention related to the present invention.

FIG. 13 is a block diagram showing a configuration of an endoscope system according to an embodiment of a second invention related to the present invention.

FIG. 14 is a block diagram showing a configuration of an endoscope system according to an embodiment of a third invention related to the present invention.

FIG. 15 is a block diagram showing a configuration of a sensor unit included in the endoscope system according to the embodiment of the third invention related to the present invention.

FIG. 16 is a block diagram showing a configuration of an endoscope system according to an embodiment of a fourth invention related to the present invention.

FIG. 17 is a block diagram showing a configuration of an endoscope system according to an embodiment of a fifth invention related to the present invention.

FIG. 18 is a block diagram showing a configuration of an endoscope system according to an embodiment of a sixth invention related to the present invention.

### DETAILED DESCRIPTION OF THE INVENTION

Hereinafter, embodiments of the present invention will be described with reference to the drawings.

### First Embodiment

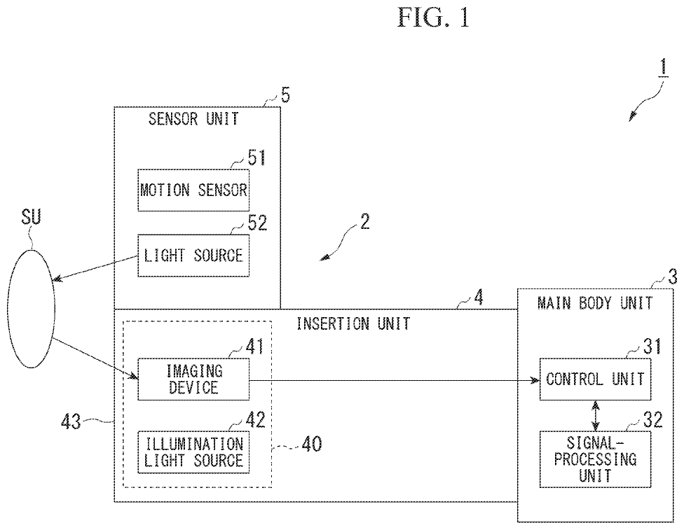

FIG. 1 shows a configuration of an endoscope system 1 according to a first embodiment of the present invention. The endoscope system 1 photographs a subject SU and generates an image of the subject SU. For example, the subject SU is an industrial product.

The endoscope system 1 shown in FIG. 1 includes an endoscope 2 and a main body unit 3. The endoscope 2 includes an insertion unit 4 and a sensor unit 5. The insertion unit 4 includes an imaging device 41 and an illumination light source 42. The sensor unit 5 includes a motion sensor 51 and a light source 52. The main body unit 3 includes a control unit 31 and a signal-processing unit 32. The size of each unit shown in FIG. 1 is different from the actual size.

A schematic configuration of the endoscope system 1 will be described. The insertion unit 4 includes a distal end part 40 disposed in the distal end of the insertion unit 4. The sensor unit 5 is disposed in the distal end part 40. The motion sensor 51 executes a measurement operation and generates a measurement value indicating a measurement result. The light source 52 generates light indicating the measurement operation at a timing that is synchronized with the measurement operation. The distal end part 40 includes the imaging device 41 that receives the light generated by the light source 52 and generates an image.

A detailed configuration of the endoscope system 1 will be described. The insertion unit 4 has an elongated (tubular) shape and is capable of bending. The insertion unit 4 is to be inserted into an object that is an observation target.

The distal end part 40 includes a distal end surface 43 of the insertion unit 4. For example, the distal end part 40 is formed with hard materials. The imaging device 41 and the illumination light source 42 are disposed in the distal end part 40.

The imaging device 41 is specifically an image sensor and, for example, is a CCD image sensor or a CMOS image sensor. The imaging device 41 includes a plurality of pixels disposed in a matrix shape and generates an image of the subject SU. Specifically, the imaging device 41 generates a moving image including two or more images (frames) at a predetermined rate (imaging rate). The imaging device 41 generates one image in each frame period. The imaging device 41 outputs the generated image to the control unit 31 of the main body unit 3.

For example, the illumination light source 42 is a light-emitting diode (LED). The illumination light source 42 generates illumination light. The light generated by the illumination light source 42 is emitted to the subject SU. The illumination light source 42 does not need to be disposed in the insertion unit 4. The illumination light source 42 may be disposed in the main body unit 3. The illumination light source 42 disposed in the main body unit 3 may generate light, and the light may be led to the distal end part 40 by a light guide such as an optical fiber disposed in the insertion unit 4.

The sensor unit 5 is separated from the insertion unit 4. The sensor unit 5 is disposed on the surface of the insertion unit 4. The sensor unit 5 is in contact with the distal end part 40. The sensor unit 5 is to be inserted into an object of an observation target along with the insertion unit 4.

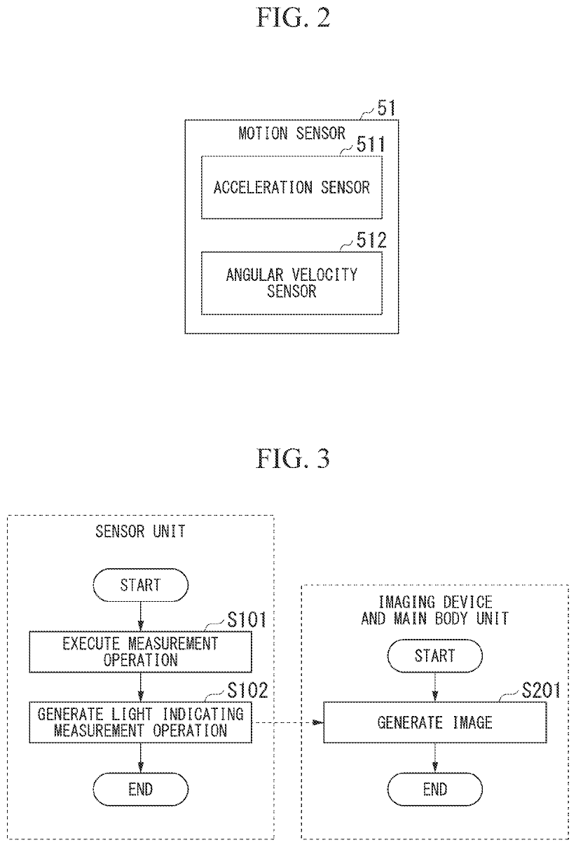

The motion sensor 51 is disposed at a position close to the imaging device 41. For example, the motion sensor 51 is an inertial sensor such as an inertial measurement unit (IMU) capable of measuring an angular velocity and an acceleration. FIG. 2 shows a configuration of the motion sensor 51. The motion sensor 51 shown in FIG. 2 includes an acceleration sensor 511 and an angular velocity sensor 512.

The acceleration sensor 511 and the angular velocity sensor 512 execute the measurement operation and generate a measurement value. For example, the acceleration sensor 511 measures a three-dimensional acceleration that occurs in the distal end part 40. The measurement value generated by the acceleration sensor 511 indicates an acceleration. A velocity can be obtained by integrating an acceleration, and a position can be obtained by integrating the velocity. For example, the angular velocity sensor 512 is a gyro sensor and measures a three-dimensional angular velocity that occurs in the distal end part 40. The measurement value generated by the angular velocity sensor 512 indicates an angular velocity. A rotation amount can be obtained by integrating an angular velocity.

The state of each of the acceleration sensor 511 and the angular velocity sensor 512 is any one of a measurement state and a stoppage state. The acceleration sensor 511 and the angular velocity sensor 512 are capable of switching between the measurement state and the stoppage state. When the state of the acceleration sensor 511 or the angular velocity sensor 512 is the measurement state, the acceleration sensor 511 or the angular velocity sensor 512 executes the measurement operation. When the state of the acceleration sensor 511 or the angular velocity sensor 512 is the stoppage state, the acceleration sensor 511 or the angular velocity sensor 512 has stopped the measurement operation.

The acceleration sensor 511 and the angular velocity sensor 512 are capable of switching between states independently of each other. The acceleration sensor 511 and the angular velocity sensor 512 do not need to simultaneously start the measurement operation and do not need to simultaneously stop the measurement operation.

For example, the light source 52 is an LED. The light source 52 generates light indicating a timing at which the measurement operation is executed. The light generated by the light source 52 is emitted toward the front of the distal end surface 43 of the insertion unit 4. The light is reflected by the subject SU and is incident on the imaging device 41.

The state of the light source 52 is any one of a light-emission state and a stoppage state. The light source 52 is capable of switching between the light-emission state and the stoppage state. When the state of the light source 52 is the light-emission state, the light source 52 generates light. When the state of the light source 52 is the stoppage state, the light source 52 has stopped light-emission.

The light source 52 is synchronized with the motion sensor 51. For example, the sensor unit 5 includes a clock generator not shown in FIG. 1, and the clock generator outputs a clock to the motion sensor 51 and the light source 52.

The motion sensor 51 and the light source 52 are capable of operating alone without receiving a control signal from the main body unit 3. Therefore, the sensor unit 5 and the main body unit 3 do not need to be electrically connected to each other, and a clock is not shared by the sensor unit 5 and the main body unit 3.

At least one of the motion sensor 51 and the light source 52 may be disposed in the distal end part 40. For example, in a case in which the distal end part 40 has two cylindrical surfaces having different diameters, the motion sensor 51 or the light source 52 may be disposed inside the inner cylindrical surface. Even in this case, the motion sensor 51 and the light source 52 are capable of operating alone without receiving a control signal from the main body unit 3. Therefore, a configuration for electrically connecting the main body unit 3 and the sensor unit 5 together does not need to be provided in the sensor unit 5.

The control unit 31 controls circuits disposed in each of the main body unit 3 and the insertion unit 4. The control unit 31 receives the image output from the imaging device 41 and outputs the image to the signal-processing unit 32. The signal-processing unit 32 processes the image output from the control unit 31 and notifies the control unit 31 of a processing result.

The control unit 31 and the signal-processing unit 32 may be constituted by at least one of a processor and a logic circuit. For example, the processor is at least one of a central processing unit (CPU), a digital signal processor (DSP), and a graphics-processing unit (GPU). For example, the logic circuit is at least one of an application-specific integrated circuit (ASIC) and a field-programmable gate array (FPGA). The control unit 31 and the signal-processing unit 32 may include one or a plurality of processors. The control unit 31 and the signal-processing unit 32 may include one or a plurality of logic circuits.

A power source is not shown in FIG. 1. For example, the main body unit 3 includes a power source that supplies power to the control unit 31, the signal-processing unit 32, the imaging device 41, and the illumination light source 42. The sensor unit 5 may include a power source that supplies power to the motion sensor 51 and the light source 52. The sensor unit 5 may be connected to the main body unit 3 by a cable, and power may be supplied from a power source of the main body unit 3 to the sensor unit 5 via the cable. The sensor unit 5 may be connected to a power source device independent of the main body unit 3 by a cable, and power may be supplied from the power source device to the sensor unit 5 via the cable. The cable is connected to the main body unit 3 by using a USB connection or the like and performs data transfer and power supply. It is assumed that synchronization of a clock is performed by using a communication method (USB, LAN, or the like) in which software functions and by transferring a signal used for synchronization of a clock by a cable. However, there is a case in which an unexpected delay occurs in accordance with the environment in which software is executed. The environment indicates a state of a CPU or a state of other software executed in parallel with the software executed in the environment. Therefore, the quality of synchronization deteriorates compared to a case in which synchronization of a clock is performed by using only hardware.

FIG. 3 shows a procedure of an operation of the endoscope system 1. The processing executed by the sensor unit 5 and the processing executed by the imaging device 41 are shown in FIG. 3.

The motion sensor 51 executes the measurement operation and generates a measurement value indicating a measurement result (Step S101). The light source 52 generates light indicating the measurement operation at a timing that is synchronized with the measurement operation (Step S102). Step S102 is executed at a timing at which Step S101 is executed. The motion sensor 51 repeats the measurement operation (Step S101). While the motion sensor 51 executes the measurement operation, the light source 52 does not need to generate light at all times.

On the other hand, the imaging device 41 receives the light generated by the light source 52 and generates an image (Step S201). The imaging device 41 repeats generation (Step S201) of an image. Since the light source 52 does not always generate light, the light is not always seen in the image.

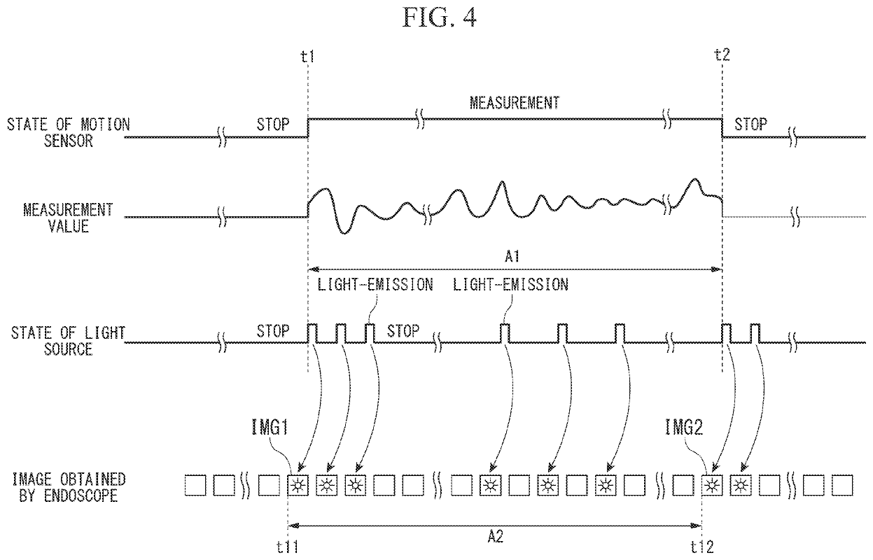

FIG. 4 shows timings of the operation of the endoscope system 1. The state of the motion sensor 51, the measurement value of the motion sensor 51, the state of the light source 52, and the image obtained by the endoscope 2 are shown in FIG. 4. The measurement value of the acceleration sensor 511 is shown as an example of the measurement value of the motion sensor 51. The horizontal direction in FIG. 4 indicates time.

For example, the state of the motion sensor 51 corresponds to the state of a control signal supplied to the motion sensor 51. When the state of the control signal is a first state, the state of the motion sensor 51 is the measurement state. For example, when the voltage of the control signal is a high level, the state of the motion sensor 51 is the measurement state. When the state of the control signal is a second state, the state of the motion sensor 51 is the stoppage state. For example, when the voltage of the control signal is a low level, the state of the motion sensor 51 is the stoppage state.

For example, the state of the light source 52 corresponds to the state of a control signal supplied to the light source 52. When the state of the control signal is a first state, the state of the light source 52 is the light-emission state. For example, when the current value of the control signal is a predetermined value greater than zero, the state of the light source 52 is the light-emission state. When the state of the control signal is a second state, the state of the light source 52 is the stoppage state. For example, when the current value of the control signal is zero, the state of the light source 52 is the stoppage state.

Before a time point t1 shown in FIG. 4 and after a time point t2 shown in FIG. 4, the state of the motion sensor 51 is the stoppage state. The state of the motion sensor 51 is the measurement state in a period A1 from the time point t1 to the time point t2. The motion sensor 51 starts the measurement operation at the time point t1 and stops the measurement operation at the time point t2. The motion sensor 51 starts generation of the measurement value at the time point t1 and stops the generation of the measurement value at the time point t2. The motion sensor 51 executes the measurement operation and generates the measurement value in the period A1.

When a predetermined time has elapsed from the timing at which the power source of the motion sensor 51 is turned on, the motion sensor 51 starts the measurement operation. For example, the length of the predetermined time is 30 seconds.

Before the time point t1, the state of the light source 52 is the stoppage state. When the motion sensor 51 starts the measurement operation at the time point t1, the light source 52 generates light indicating that the measurement operation has been started. In other words, the light source 52 generates light in a pattern corresponding to a start of the measurement operation. In the example shown in FIG. 4, the light source 52 generates light three times at timings in accordance with a frame rate of images. In other words, the light source 52 generates pulse light at a timing corresponding to each of three consecutive frame periods. The period in which the light source 52 continues light-emission is shorter than one frame period. The light source 52 starts light-emission in one frame period and stops the light-emission in the same frame period.

While the motion sensor 51 executes the measurement operation, the light source 52 generates light indicating that the measurement operation continues. In other words, the light source 52 generates light in a pattern corresponding to continuation of the measurement operation. In the example shown in FIG. 4, the light source 52 generates pulse light at a timing corresponding to the first frame period of two consecutive frame periods and stops light-emission at a timing corresponding to the next frame period. The light source 52 repeats this operation until the motion sensor 51 stops the measurement operation.

When the motion sensor 51 stops the measurement operation at the time point t2, the light source 52 generates light indicating that the measurement operation is stopped. In other words, the light source 52 generates light in a pattern corresponding to a stoppage of the measurement operation. In the example shown in FIG. 4, the light source 52 generates light twice at timings in accordance with the frame rate of images. In other words, the light source 52 generates pulse light at a timing corresponding to each of two consecutive frame periods. Thereafter, the state of the light source 52 is the stoppage state.

The imaging device 41 is disposed at a position reached by light emitted from the light source 52 and reflected by the subject SU. The imaging device 41 generates an image in which the light is seen. In this way, the timing of the measurement operation executed by the motion sensor 51 is recorded in the image. For example, the light is seen in a predetermined region in the image.

When the motion sensor 51 starts the measurement operation, the light source 52 generates light three times. The light generated by the light source 52 is seen in images of three consecutive frame periods. The time point t1 at which the motion sensor 51 starts the measurement operation corresponds to a time point t11 at which an image IMG1 is generated in the first frame period of the three frame periods.

When the motion sensor 51 stops the measurement operation, the light source 52 generates light twice. The light generated by the light source 52 is seen in images of two consecutive frame periods. The time point t2 at which the motion sensor 51 stops the measurement operation corresponds to a time point t12 at which an image IMG2 is generated in the first frame period of the two frame periods.

Therefore, it is possible to associate the period A1 during which the motion sensor 51 continues the measurement operation and a period A2 detected on the basis of the image in which light is seen with each other. In other words, it is possible to synchronize the measurement value generated in the period A1 and the image generated in the period A2 with each other. There is a possibility that the length of any one of the period A1 and the period A2 is shifted from a correct value. In such a case, the endoscope system 1 may correct time points on the basis of the correct one of the period A1 and the period A2 and then may associate the period A1 and the period A2 with each other.

In the example shown in FIG. 4, after the motion sensor 51 starts or stops the measurement operation, the light source 52 generates light in two or more consecutive frame periods including a first frame period and a second frame period. The light source 52 generates light in the first frame period. After the light source 52 stops light-emission in the first frame period, the light source 52 generates light in the second frame period following the first frame period. The light source 52 stops light-emission in the second frame period.

The start timing of the exposure period of the imaging device 41 does not necessarily match the timing at which the light source 52 starts light-emission. In a case in which the light source 52 generates light in a short period, there is a possibility that the period is not included in the exposure period of the imaging device 41. Therefore, the light source 52 may generate light in the longest possible period shorter than one frame period. For example, the light source 52 may generate light in a longer period than half the exposure period of the imaging device 41.

In the example shown in FIG. 4, the light source 52 generates light in a shorter period than one frame period. Since the light source 52 intermittently generates light, an effect of saving the power consumption of the light source 52 can be obtained. In addition, the relative amount of light of the light source 52 to that of the illumination light source 42 can be reduced. Therefore, the influence caused by the light source 52 on the image generated by the imaging device 41 can be suppressed and an effect of facilitating observation can be obtained.

The light source 52 is capable of switching between a light-emission state (first state) in which the light source 52 generates light and a stoppage state (second state) in which the light source 52 stops light-emission. The pattern in which the light-emission state occurs corresponds to the state of the measurement operation. For example, the pattern is shown by at least one of the number of light-emission states and the length of the light-emission state.

The light source 52 generates light at a timing (first timing) at which the motion sensor 51 starts the measurement operation. In the example shown in FIG. 4, the light source 52 starts light-emission at the time point t1 at which the motion sensor 51 starts the measurement operation. The light source 52 generates light three times at the same rate as the frame rate of images. In this case, the light-emission state occurs three times.

The light source 52 generates light at a timing (second timing) at which the motion sensor 51 stops the measurement operation. In the example shown in FIG. 4, the light source 52 starts light-emission at the time point t2 at which the motion sensor 51 stops the measurement operation. The light source 52 generates light twice at the same rate as the frame rate of images. In this case, the light-emission state occurs twice.

The number of periods during which the light-emission state occurs corresponds to the state of the measurement operation. The length of the period is the same as that of the frame period. In the example shown in FIG. 4, the light-emission state indicating a start of the measurement operation occurs in three consecutive periods. In the example shown in FIG. 4, the light-emission state indicating a stoppage of the measurement operation occurs in two consecutive periods.

The number of periods during which the light-emission state continues may correspond to the state of the measurement operation. In such a case, the light source 52 continuously generates light in a longer period than one frame period. For example, the light-emission state indicating the start of the measurement operation may continue in three consecutive periods. The light-emission state indicating the stoppage of the measurement operation may continue in two consecutive periods. In this way, a similar effect to that of the case in which the light source 52 intermittently generates light in each of three or two frame periods can be obtained.

While the motion sensor 51 executes the measurement operation, the light source 52 generates light indicating that the measurement operation continues. In the example shown in FIG. 4, the light source 52 generates light at half the frame rate of images. Therefore, the frame period during which the light source 52 generates light and the frame period during which the light source 52 stops light-emission alternately occur. In this case, the light-emission state periodically occurs.

The light source 52 may generate light only when the motion sensor 51 starts the measurement operation. The light source 52 does not need to generate light when the measurement operation is stopped. Since the light source 52 generates light indicating the start of the measurement operation, it is possible to associate the time point t1 at which the motion sensor 51 starts the measurement operation and the time point t11 at which the image IMG1 in which the light is seen is generated with each other. In other words, it is possible to associate the time point t1 at which generation of measurement values is started and the time point t11 at which the image IMG1 is generated with each other.

The motion sensor 51 starts generation of measurement values at the time point t1 and stops the generation of measurement values at the time point t2. First time-point information indicating a time point at which a measurement value is generated is attached to the measurement value. It is possible to calculate the length of the period A1 from the time point t1 to the time point t2 on the basis of the time point t1 indicated by the first time-point information and the time point t2 indicated by the first time-point information. Second time-point information indicating a time point at which an image is generated is attached to the image. It is possible to calculate the time point t12 at which the image IMG2 is generated on the basis of both the time point t11 indicated by the second time-point information attached to the image IMG1 and the length of the period A1. In this way, it is possible to associate the time point t2 at which the generation of measurement values is stopped and the time point t12 at which the image IMG2 is generated with each other. Therefore, the light source 52 does not need to generate light indicating the stoppage of the measurement operation.

The light source 52 does not need to generate the light indicating that the measurement operation continues. In a case in which the light is seen in an image, it is possible to confirm that loss of measurement has not occurred. After the light source 52 stops generation of the light indicating continuation of the measurement operation, the light source 52 does not need to generate the light indicating the stoppage of the measurement operation.

The light source 52 may generate light when the acceleration sensor 511 starts the measurement operation and may generate light when the angular velocity sensor 512 starts the measurement operation. The light source 52 may generate light when the acceleration sensor 511 stops the measurement operation and may generate light when the angular velocity sensor 512 stops the measurement operation. The light source 52 may generate light indicating that the measurement operation executed by the acceleration sensor 511 continues and may generate light indicating that the measurement operation executed by the angular velocity sensor 512 continues. The pattern of light indicating the state of the measurement operation executed by the acceleration sensor 511 and the pattern of light indicating the state of the measurement operation executed by the angular velocity sensor 512 are different from each other.

In the example shown in FIG. 4, the light source 52 generates light having a pattern of the light-emission state corresponding to the state of the measurement operation. The sensor unit 5 does not need to include two or more light sources. Therefore, the sensor unit 5 becomes lightweight.

For example, the light source 52 generates visible light. Since the light source 52 generates similar visible light to that generated by the illumination light source 42 for observation, special design change or special processing is not necessary in the imaging device 41 or the like. A user can confirm light seen in an image. The light source 52 may generate visible light having a predetermined color. For example, the light source 52 may generate red light. In this case, a user can easily confirm light seen in an image.

The light source 52 may generate one of infrared light and ultraviolet light. In a case in which the light source 52 generates infrared light, the imaging device 41 may include pixels sensitive to infrared light. In a case in which the light source 52 generates ultraviolet light, the imaging device 41 may include pixels sensitive to ultraviolet light. In a case in which the light source 52 generates one of infrared light and ultraviolet light, such light is not seen by a human Therefore, such light does not disturb observation performed by a user.

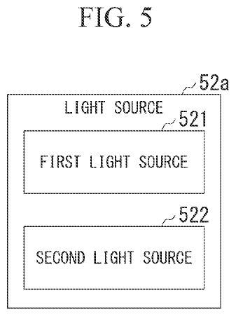

The light source 52 may generate light having wavelengths corresponding to the state of the measurement operation. For example, the light source 52 may be changed to a light source 52a shown in FIG. 5. The light source 52a includes a first light source 521 and a second light source 522. The first light source 521 generates light having a first wavelength at a timing at which the motion sensor 51 starts the measurement operation. The second light source 522 generates light having a second wavelength different from the first wavelength at a timing at which the motion sensor 51 stops the measurement operation. The imaging device 41 includes pixels sensitive to the light having the first wavelength and includes pixels sensitive to the light having the second wavelength.

The light source 52a may include a light source configured to generate light that indicates continuation of the measurement operation and has a third wavelength. The third wavelength is different from the first wavelength and the second wavelength. The imaging device 41 may include pixels sensitive to the light having the third wavelength.

In the first embodiment, the endoscope system 1 can synchronize a measurement value generated by the motion sensor 51 and an image generated by the imaging device 41 with each other.

Since the sensor unit 5 and the main body unit 3 are not electrically connected to each other, a signal line or the like for sharing a clock between the sensor unit 5 and the main body unit 3 is not necessary. Since the light generated by the light source 52 is recorded in an image, no delay due to wireless communication or the like occurs between a timing at which the light is generated and a timing at which the image is generated.

### Second Embodiment

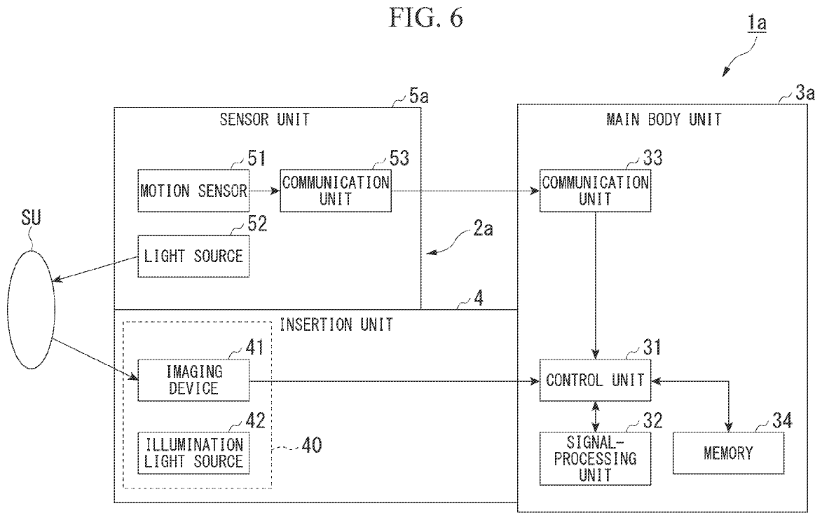

FIG. 6 shows a configuration of an endoscope system 1a according to a second embodiment of the present invention. The same configuration as that shown in FIG. 1 will not be described.

The endoscope 2 shown in FIG. 1 is changed to an endoscope 2a. The main body unit 3 shown in FIG. 1 is changed to a main body unit 3a. The main body unit 3a includes a communication unit 33 and a memory 34 in addition to the control unit 31 and the signal-processing unit 32 shown in FIG. 1. The sensor unit 5 shown in FIG. 1 is changed to a sensor unit 5a. The sensor unit 5a includes a communication unit 53 in addition to the motion sensor 51 and the light source 52 shown in FIG. 1.

The motion sensor 51 generates a measurement value and outputs the measurement value to the communication unit 53. The communication unit 53 includes a communication circuit and is connected to the communication unit 33 of the main body unit 3a wirelessly or by a cable. The communication unit 53 transmits the measurement value output from the motion sensor 51 to the communication unit 33. In a case in which the communication unit 53 and the communication unit 33 are connected to each other by a cable, a USB cable or the like is used. As described above, in a case in which synchronization of a clock is performed by using a communication method in which software functions and by transferring a signal used for synchronization of a clock by a cable, there is a possibility that the quality of synchronization deteriorates compared to a case in which synchronization of a clock is performed by using only hardware.

While the motion sensor 51 executes the measurement operation, the communication unit 53 may transmit the measurement value to the communication unit 33. The sensor unit 5a may include a memory that stores the measurement value output from the motion sensor 51. After the motion sensor 51 stops the measurement operation, the communication unit 53 may transmit the measurement value stored on the memory to the communication unit 33.

The communication unit 33 includes a communication circuit and is connected to the communication unit 53 of the sensor unit 5a wirelessly or by a cable. The communication unit 33 receives the measurement value transmitted by the communication unit 53 and outputs the measurement value to the control unit 31.

The control unit 31 outputs the image output from the imaging device 41 and the measurement value received by the communication unit 33 to the signal-processing unit 32. The signal-processing unit 32 detects the light generated by the light source 52 by processing the image. The signal-processing unit 32 associates a time point at which the measurement operation is executed and a time point at which the image is generated with each other so as to associate the measurement value and the image with each other.

The signal-processing unit 32 outputs the measurement value and the image associated with each other to the control unit 31. The control unit 31 outputs the measurement value and the image to the memory 34. The memory 34 is a volatile or nonvolatile memory. For example, the memory 34 is at least one of a random-access memory (RAM), a dynamic random-access memory (DRAM), a static random-access memory (SRAM), a read-only memory (ROM), an erasable programmable read-only memory (EPROM), an electrically erasable programmable read-only memory (EEPROM), and a flash memory. The memory 34 stores the measurement value and the image associated with each other.

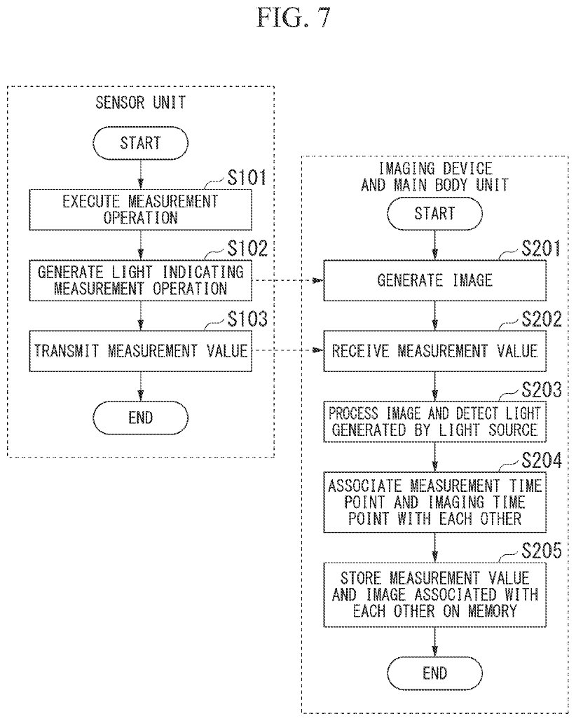

FIG. 7 shows a procedure of an operation of the endoscope system 1a. The processing executed by the sensor unit 5a and the processing executed by the imaging device 41 and the main body unit 3a are shown in FIG. 7. The same processing as that shown in FIG. 3 will not be described.

The motion sensor 51 generates a measurement value in Step S101. The light source 52 generates light indicating the measurement operation in Step S102. After Step S101 and Step S102, the communication unit 53 transmits the measurement value generated by the motion sensor 51 to the communication unit 33 of the main body unit 3a (Step S103). In a case in which the measurement operation and transmission of the measurement value are simultaneously executed, Steps S101 to S103 are repetitively executed.

After the imaging device 41 generates an image in Step S201, the communication unit 33 receives the measurement value transmitted by the communication unit 53 of the sensor unit 5a (Step S202). In a case in which the measurement operation and transmission of the measurement value are simultaneously executed, generation of an image and reception of the measurement value are simultaneously executed. Therefore, Step S201 and Step S202 are repetitively executed.

After Step S201 and Step S202, the signal-processing unit 32 detects light generated by the light source 52 by processing the image generated by the imaging device 41 (Step S203). The light source 52 generates light having a pattern of the light-emission state corresponding to the state of the measurement operation. The signal-processing unit 32 analyzes the pattern in Step S203.

For example, the light source 52 generates light at a first timing at which the motion sensor 51 starts the measurement operation. The light indicates a start of the measurement operation. The first timing corresponds to the time point t1 shown in FIG. 4. The signal-processing unit 32 detects light generated at the first timing by the light source 52. In the example shown in FIG. 4, when the motion sensor 51 starts the measurement operation, the light source 52 generates light three times. The light generated by the light source 52 is seen in images of three consecutive frame periods. When light is detected in three consecutive images, the signal-processing unit 32 detects light generated at the first timing.

The light source 52 generates light at a second timing at which the motion sensor 51 stops the measurement operation. The light indicates a stoppage of the measurement operation. The second timing corresponds to the time point t2 shown in FIG. 4. The signal-processing unit 32 detects light generated at the second timing by the light source 52. In the example shown in FIG. 4, when the motion sensor 51 stops the measurement operation, the light source 52 generates light twice. The light generated by the light source 52 is seen in images of two consecutive frame periods. When light is detected in two consecutive images, the signal-processing unit 32 detects light generated at the second timing.

While the motion sensor 51 executes the measurement operation, the light source 52 generates light indicating that the measurement operation continues. The signal-processing unit 32 detects the light indicating that the measurement operation continues by processing the image output from the imaging device 41. In the example shown in FIG. 4, the frame period during which the light source 52 generates light and the frame period during which the light source 52 stops light-emission alternately occur. The signal-processing unit 32 detects the light indicating that the measurement operation continues in a case in which light is detected in one of two consecutive images, light is not detected in the other of the two images, and such a state continues in four or more frame periods.

After the signal-processing unit 32 detects the light generated by the light source 52, the signal-processing unit 32 associates a time point (measurement time point) at which the measurement operation is executed and a time point (imaging time point) at which the image in which the light is seen is generated with each other. In this way, the signal-processing unit 32 associates the measurement value and the image with each other (Step S204).

For example, the signal-processing unit 32 extracts a time point at which the image is generated in which the light generated at the first timing by the light source 52 is seen. In the example shown in FIG. 4, the signal-processing unit 32 extracts the time point t11 from the image IMG1. The signal-processing unit 32 extracts a time point of the measurement value first generated at the timing at which the motion sensor 51 starts the measurement operation. In the example shown in FIG. 4, the signal-processing unit 32 extracts the time point t1 from the measurement value. The signal-processing unit 32 calculates the difference between a time point of each measurement value and a time point of the first generated measurement value. The signal-processing unit 32 replaces the time point of the first generated measurement value with the time point extracted from the image. The signal-processing unit 32 corrects the time point of each measurement value on the basis of the above-described difference. The signal-processing unit 32 associates the measurement value and the image with each other by executing the above-described processing.

The signal-processing unit 32 may correct the time point of the image on the basis of the time point of the measurement value. For example, the signal-processing unit 32 may replace the time point of the image corresponding to the first generated measurement value with the time point of the measurement value.

After the measurement value and the image are associated with each other, the control unit 31 stores the measurement value and the image on the memory 34 (Step S205).

After the motion sensor 51 stops the measurement operation, the communication unit 53 may transmit the measurement value to the communication unit 33. The signal-processing unit 32 may execute Step S203 and Step S204 before the communication unit 33 receives the measurement value.

When the signal-processing unit 32 detects light indicating the start of the measurement operation, the control unit 31 may display information indicating the start of the measurement operation on a display unit not shown in FIG. 1. Alternatively, the control unit 31 may cause a speaker not shown in FIG. 1 to generate a voice indicating the start of the measurement operation. In this way, the control unit 31 may notify a user of the start of the measurement operation.

When a predetermined period of time elapses from a timing at which a power source of the motion sensor 51 is turned on, the motion sensor 51 starts the measurement operation. In a case in which the predetermined period of time elapses and a user is not notified of the start of the measurement operation, the user may turn off the power source of the motion sensor 51 and may turn on the power source of the motion sensor 51 again.

In the second embodiment, the endoscope system 1a can connect together a measurement value and an image having time points synchronized with each other. The sensor unit 5a is electrically connected to the main body unit 3a. However, a signal line or the like for sharing a clock between the sensor unit 5a and the main body unit 3a is not necessary.

### Third Embodiment

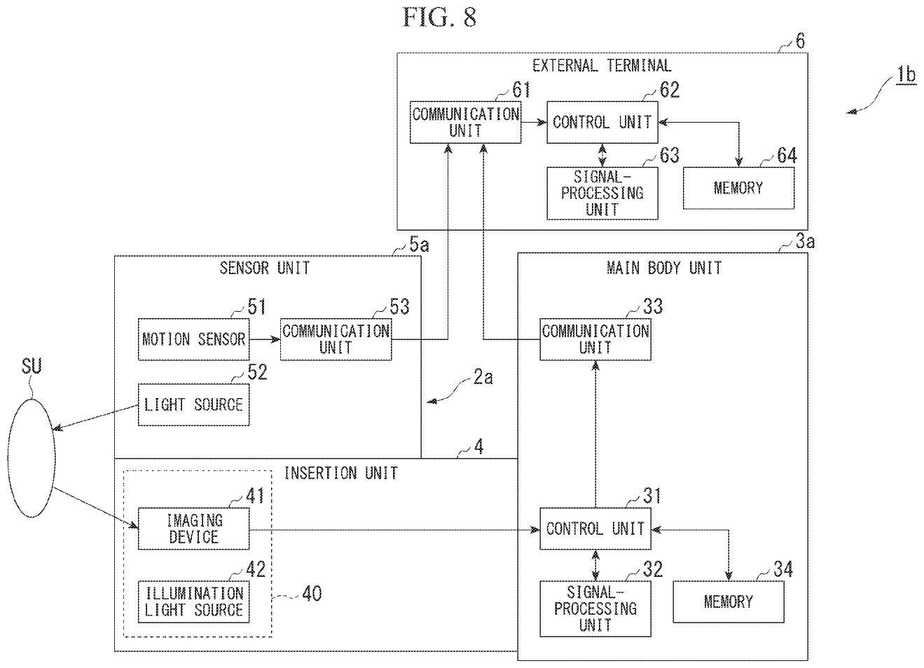

FIG. 8 shows a configuration of an endoscope system 1b according to a third embodiment of the present invention. The same configuration as that shown in FIG. 6 will not be described.

The endoscope system 1b includes an external terminal 6 in addition to the main body unit 3a, the insertion unit 4, and the sensor unit 5a shown in FIG. 6. The external terminal 6 includes a communication unit 61, a control unit 62, a signal-processing unit 63, and a memory 64.

The communication unit 53 of the sensor unit 5a transmits a measurement value output from the motion sensor 51 to the communication unit 61 of the external terminal 6.

The control unit 31 of the main body unit 3a outputs an image processed by the signal-processing unit 32 to the communication unit 33. The communication unit 33 transmits the image output from the control unit 31 to the communication unit 61 of the external terminal 6.

The communication unit 61 of the external terminal 6 includes a communication circuit and is connected to the communication unit 53 of the sensor unit 5a and the communication unit 33 of the main body unit 3a wirelessly or by a cable. The communication unit 61 receives the measurement value transmitted by the communication unit 53 and receives the image transmitted by the communication unit 33.

The communication unit 61 outputs the received measurement value and the received image to the control unit 62. The communication unit 61 may include a first communication unit that receives the measurement value and a second communication unit that receives the image.

The control unit 62 outputs the measurement value and the image received by the communication unit 61 to the signal-processing unit 63. The signal-processing unit 63 has a similar function to that of the signal-processing unit 32 in the second embodiment. The signal-processing unit 63 detects light generated by the light source 52 by processing the image. The signal-processing unit 63 associates a time point at which the measurement operation is executed and a time point at which the image is generated with each other so as to associate the measurement value and the image with each other.

The signal-processing unit 63 outputs the measurement value and the image associated with each other to the control unit 62. The control unit 62 outputs the measurement value and the image to the memory 64. The memory 64 is a volatile or nonvolatile memory. For example, the memory 64 is at least one of a RAM, a DRAM, a SRAM, a ROM, an EPROM, an EEPROM, and a flash memory. The memory 64 stores the measurement value and the image associated with each other.

The control unit 62 and the signal-processing unit 63 may be constituted by at least one of a processor and a logic circuit. The control unit 62 and the signal-processing unit 63 may include one or a plurality of processors. The control unit 62 and the signal-processing unit 63 may include one or a plurality of logic circuits.

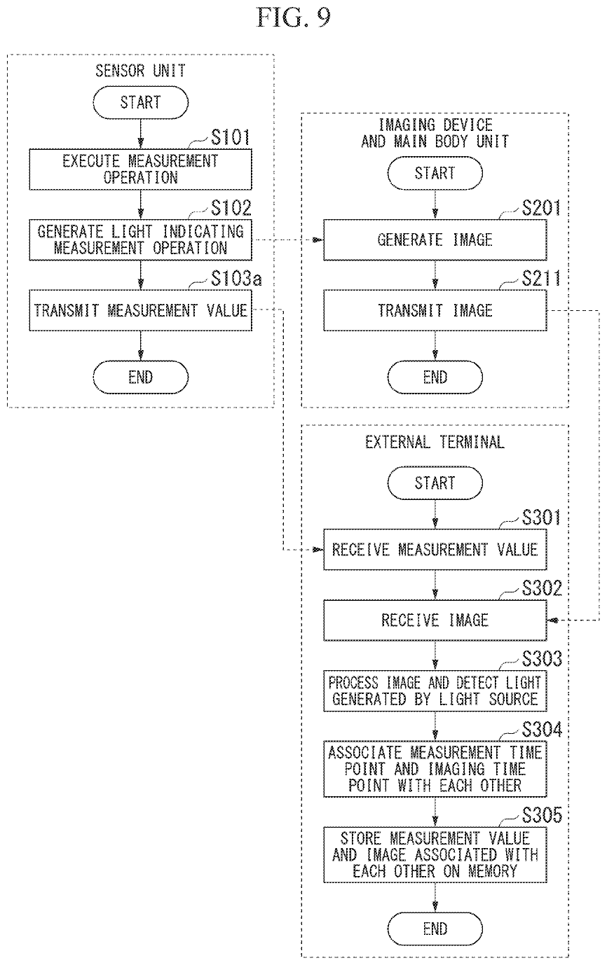

FIG. 9 shows a procedure of an operation of the endoscope system 1b. The processing executed by the sensor unit 5a, the processing executed by the imaging device 41 and the main body unit 3a, and the processing executed by the external terminal 6 are shown in FIG. 9. The same processing as that shown in FIG. 3 will not be described.

The motion sensor 51 generates a measurement value in Step S101. The light source 52 generates light indicating the measurement operation in Step S102. After Step S101 and Step S102, the communication unit 53 transmits the measurement value generated by the motion sensor 51 to the communication unit 61 of the external terminal 6 (Step S103a). In a case in which the measurement operation and transmission of the measurement value are simultaneously executed, Steps S101 to S103a are repetitively executed.

After the imaging device 41 generates an image in Step S201, the communication unit 33 transmits the image to the communication unit 61 of the external terminal 6 (Step S211). While the imaging device 41 continuously generates an image, the image may be accumulated on the memory 34. The communication unit 33 may transmit the image to the communication unit 61 at a predetermined timing. In a case in which generation of the image and transmission of the image are simultaneously executed, Step S201 and Step S211 are repetitively executed.

The communication unit 61 of the external terminal 6 receives the measurement value transmitted by the communication unit 53 of the sensor unit 5a (Step S301) and receives the image transmitted by the communication unit 33 of the main body unit 3a (Step S302). The communication unit 61 may receive the image after receiving the measurement value. Alternatively, the communication unit 61 may receive the measurement value after receiving the image. In a case in which the measurement operation and transmission of the measurement value are simultaneously executed and generation of the image and transmission of the image are simultaneously executed, reception of the measurement value and reception of the image may be simultaneously executed.

After Step S301 and Step S302, the signal-processing unit 63 detects light generated by the light source 52 by processing the image received by the communication unit 61 (Step S303). Step S303 is similar to Step S203 shown in FIG. 7.

After Step S303, the signal-processing unit 63 associates a time point (measurement time point) at which the measurement operation is executed and a time point (imaging time point) at which the image in which the light is seen is generated with each other. In this way, the signal-processing unit 63 associates the measurement value and the image with each other (Step S304). Step S304 is similar to Step S204 shown in FIG. 7.

After Step S304, the control unit 62 stores the measurement value and the image on the memory 64 (Step S305). Step S305 is similar to Step S205 shown in FIG. 7.

After the motion sensor 51 stops the measurement operation, the communication unit 53 may transmit the measurement value to the communication unit 61. The signal-processing unit 63 may execute Step S303 and Step S304 before the communication unit 61 receives the measurement value.

In the third embodiment, the endoscope system 1b can connect together a measurement value and an image having time points synchronized with each other. Since the sensor unit 5a and the main body unit 3a are not electrically connected to each other, a signal line or the like for sharing a clock between the sensor unit 5a and the main body unit 3a is not necessary.

### Fourth Embodiment

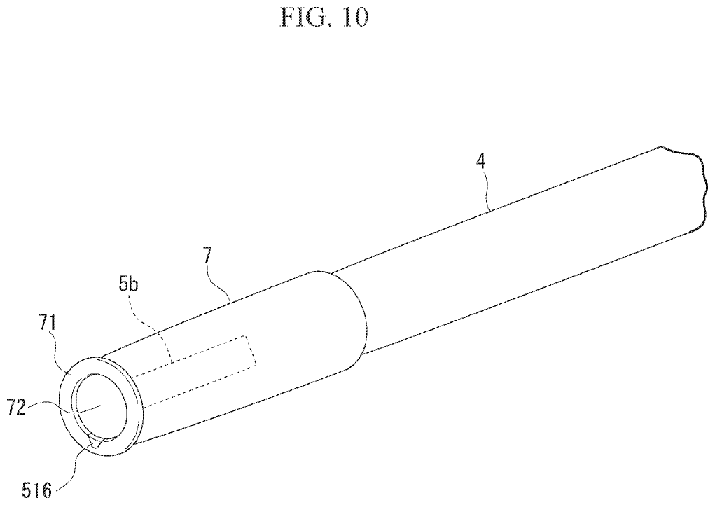

The endoscope system according to a fourth embodiment of the present invention includes the main body unit 3 and the insertion unit 4 shown in FIG. 1 and an optical adaptor 7 shown in FIG. 10. FIG. 10 shows a configuration of the distal end of the insertion unit 4. Various adaptors for an endoscope can be connected to the distal end of the insertion unit 4. In the example shown in FIG. 10, the optical adaptor 7 for observation using an endoscope is connected to the distal end of the insertion unit 4.

The optical adaptor 7 includes a sensor unit 5b. The distal end part 40 shown in FIG. 1 is disposed at the distal end of the insertion unit 4. The optical adaptor 7 is attachable to and detachable from the distal end part 40. Therefore, the sensor unit 5b is attachable to and detachable from the distal end part 40.

The optical adaptor 7 includes an illumination light source 71 and an LED 516. An opening portion 72 for capturing light in the optical adaptor 7 is formed at the distal end of the optical adaptor 7. The illumination light source 71 and the LED 516 are disposed around the opening portion 72. The illumination light source 71 generates illumination light emitted to a subject. The LED 516 generates light indicating a timing at which the measurement operation is executed. The direction in which the LED 516 emits light does not need to match an imaging direction.

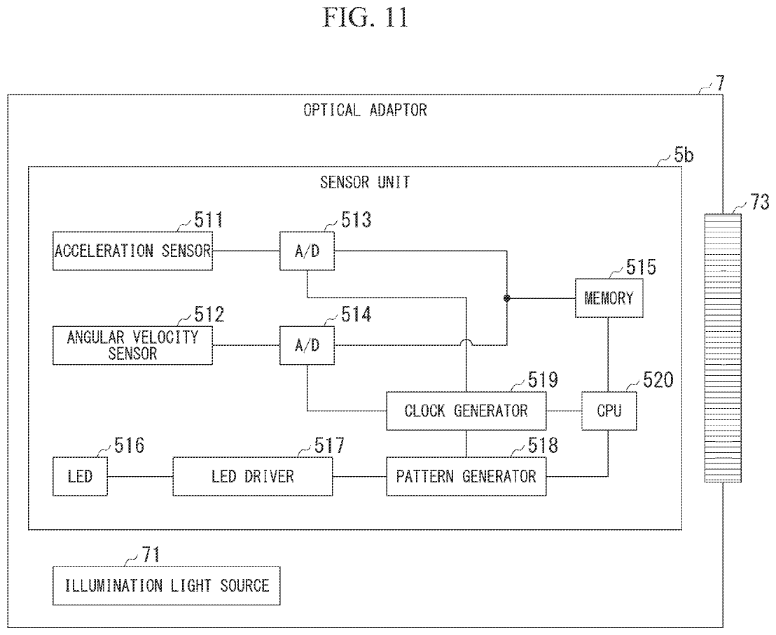

FIG. 11 shows a configuration of the optical adaptor 7. The optical adaptor 7 shown in FIG. 11 includes the sensor unit 5b, the illumination light source 71, and a connector 73. An optical system for transmitting light captured in the optical adaptor 7 to the insertion unit 4 is not shown in FIG. 11.

The sensor unit 5b includes an acceleration sensor 511, an angular velocity sensor 512, an A/D converter 513, an A/D converter 514, a memory 515, the LED 516, an LED driver 517, a pattern generator 518, a clock generator 519, and a CPU 520 (signal output circuit).

The acceleration sensor 511 is the same as the acceleration sensor 511 shown in FIG. 2. The angular velocity sensor 512 is the same as the angular velocity sensor 512 shown in FIG. 2.

The A/D converter 513 performs AD conversion on an analog measurement value output from the acceleration sensor 511 and converts the measurement value into a digital measurement value. The A/D converter 514 performs AD conversion on an analog measurement value output from the angular velocity sensor 512 and converts the measurement value into a digital measurement value. The A/D converter 513 and the A/D converter 514 execute processing in synchronization with a clock generated by the clock generator 519.

The memory 515 is a volatile or nonvolatile memory. For example, the memory 515 is at least one of a RAM, a DRAM, a SRAM, a ROM, an EPROM, an EEPROM, and a flash memory. The memory 515 stores the measurement value output from the A/D converter 513 and the measurement value output from the A/D converter 514.

The LED 516 is the same as the LED 516 shown in FIG. 10. The LED driver 517 controls the operation of the LED 516 by controlling the current output to the LED 516.

The clock generator 519 includes an oscillator and generates a clock. The CPU 520 generates a control signal indicating a start of the measurement operation or a stoppage of the measurement operation and outputs the control signal to each of the acceleration sensor 511 and the angular velocity sensor 512. The A/D converter 513 and the A/D converter 514 perform AD conversion on the basis of the control signal.

The CPU 520 outputs the above-described control signal to the pattern generator 518. The pattern generator 518 generates a pattern of the light-emission state indicating the start of the measurement operation or the stoppage of the measurement operation at a timing at which the control signal is input. The pattern generator 518 outputs a control signal corresponding to the pattern to the LED driver 517 in synchronization with the clock generated by the clock generator 519. The LED driver 517 controls the operation of the LED 516 on the basis of the control signal. The LED 516 generates light indicating the start of the measurement operation or the stoppage of the measurement operation. In this way, the LED 516 generates light at a timing indicated by the control signal output from the CPU 520 to each of the acceleration sensor 511 and the angular velocity sensor 512.

The illumination light source 71 is the same as the illumination light source 71 shown in FIG. 10. The connector 73 electrically connects together the optical adaptor 7 and the insertion unit 4. For example, the power output from the power source disposed in the main body unit 3 is supplied to the optical adaptor 7 via both a cable disposed in the insertion unit 4 and the connector 73. Alternatively, the CPU 520 performs communication with the control unit 31 of the main body unit 3 via both a cable disposed in the insertion unit 4 and the connector 73. Alternatively, a driving signal for driving the illumination light source 71 is output from the control unit 31 to the illumination light source 71 via both a cable disposed in the insertion unit 4 and the connector 73. The CPU 520 may perform filter processing such as low-pass filter processing for reducing noise, high-pass filter processing for emphasizing a specific frequency, or the like on each of the measurement value of the acceleration and the angular velocity before performing communication.

The optical adaptor 7 may include a communication unit. The communication unit may transmit the measurement value to the communication unit 33 of the main body unit 3a shown in FIG. 6 or the communication unit 61 of the external terminal 6 shown in FIG. 8.

The sensor unit 5b may be disposed in an adaptor used for an endoscope and the adaptor may be a different type from the optical adaptor.

In the fourth embodiment, the endoscope system can connect together a measurement value and an image having time points synchronized with each other. Since the sensor unit 5b is disposed in an adaptor used for an endoscope, the sensor unit 5b can be easily disposed in the distal end part 40 of the insertion unit 4.

### Embodiment of First Invention

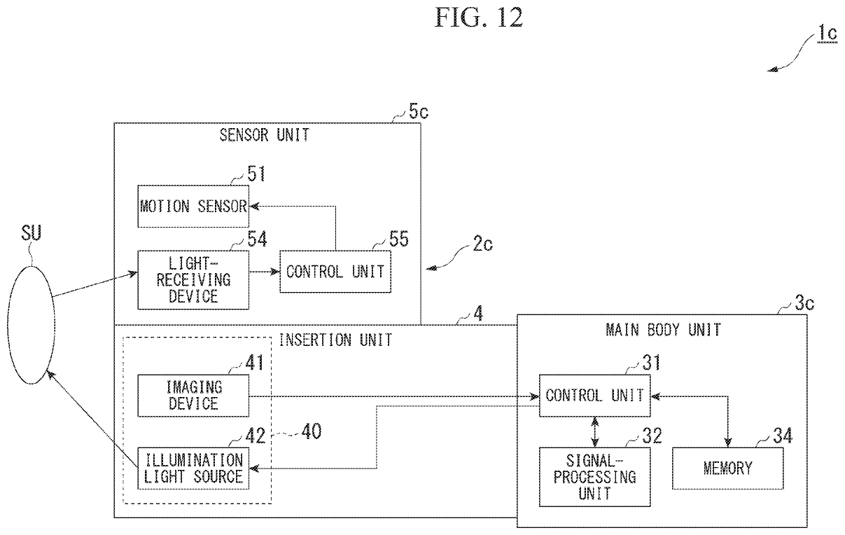

FIG. 12 shows a configuration of an endoscope system 1c according to an embodiment of a first invention related to the present invention. The same configuration as that shown in FIG. 1 will not be described.

The endoscope 2 shown in FIG. 1 is changed to an endoscope 2c. The main body unit 3 shown in FIG. 1 is changed to a main body unit 3c. The main body unit 3c includes a memory 34 in addition to the control unit 31 and the signal-processing unit 32 shown in FIG. 1. The sensor unit 5 shown in FIG. 1 is changed to a sensor unit 5c. The sensor unit 5c includes a motion sensor 51, a light-receiving device 54, and a control unit 55.

The memory 34 is a nonvolatile memory. The control unit 31 records an image output from the imaging device 41 on the memory 34. The control unit 31 instructs the illumination light source 42 to generate light indicating a start of the measurement operation at a timing at which recording of an image is started. The control unit 31 instructs the illumination light source 42 to generate light indicating a stoppage of the measurement operation at a timing at which the recording of an image is stopped.

The illumination light source 42 of the insertion unit 4 generates light for requesting the sensor unit 5c to start or stop the measurement operation. When the control unit 31 instructs the illumination light source 42 to generate the light indicating the start of the measurement operation, the illumination light source 42 generates light having a pattern of the light-emission state corresponding to the start of the measurement operation. In other words, the illumination light source 42 generates the light indicating the start of the measurement operation at a timing at which the recording of an image is started. Thereafter, when the control unit 31 instructs the illumination light source 42 to generate the light indicating the stoppage of the measurement operation, the illumination light source 42 generates light having a pattern of the light-emission state corresponding to the stoppage of the measurement operation. In other words, the illumination light source 42 generates the light indicating the stoppage of the measurement operation at a timing at which the recording of an image is stopped.

The light generated by the illumination light source 42 is reflected by the subject SU and is incident on the light-receiving device 54 of the sensor unit 5c. The light-receiving device 54 is a photodiode. The light-receiving device 54 receives the light generated by the illumination light source 42 and generates a signal in accordance with the amount of the light. The signal is converted into a digital signal by an A/D converter not shown in FIG. 12 and is output to the control unit 55.

The control unit 55 may be constituted by at least one of a processor and a logic circuit. The control unit 55 may include one or a plurality of processors. The control unit 55 may include one or a plurality of logic circuits.

The control unit 55 detects the light generated by the illumination light source 42 on the basis of the digital signal in accordance with the amount of the light incident on the light-receiving device 54. The control unit 55 analyzes a pattern of the light-emission state on the basis of the intensity of light indicated by the value of the digital signal. In this way, the control unit 55 detects the light indicating the start of the measurement operation or the stoppage of the measurement operation. When the light indicating the start of the measurement operation is detected, the control unit 55 outputs a control signal indicating the start of the measurement operation to the motion sensor 51 and causes the motion sensor 51 to start the measurement operation. When the light indicating the stoppage of the measurement operation is detected, the control unit 55 outputs a control signal indicating the stoppage of the measurement operation to the motion sensor 51 and causes the motion sensor 51 to stop the measurement operation.

When the control signal indicating the start of the measurement operation is output from the control unit 55, the motion sensor 51 starts the measurement operation. When the control signal indicating the stoppage of the measurement operation is output from the control unit 55, the motion sensor 51 stops the measurement operation.

The sensor unit 5c may include the communication unit 53 shown in FIG. 6, and the main body unit 3c may include the communication unit 33 shown in FIG. 6. In such a case, the communication unit 53 may transmit the measurement value output from the motion sensor 51 to the communication unit 33. The control unit 31 may associate the measurement value with the image recorded on the memory 34 and may record the measurement value on the memory 34.

In a case in which the sensor unit 5c includes the communication unit 53 and the main body unit 3c includes the communication unit 33, the endoscope system 1c may include the external terminal 6 shown in FIG. 8. The communication unit 53 may transmit the measurement value output from the motion sensor 51 to the communication unit 61 of the external terminal 6. The communication unit 33 may transmit the image recorded on the memory 34 to the communication unit 61 of the external terminal 6. The control unit 62 may associate the measurement value and the image received by the communication unit 61 with each other and may record the measurement value and the image on the memory 64.

The sensor unit 5c may be disposed in an adaptor used for an endoscope.

In the embodiment of the first invention, the illumination light source 42 generates light indicating the measurement operation, and the control unit 55 controls the measurement operation executed by the motion sensor 51 on the basis of the light received by the light-receiving device 54. The motion sensor 51 executes the measurement operation and generates a measurement value in a period during which an image is recorded on the memory 34. Therefore, the endoscope system 1c can synchronize a measurement value generated by the motion sensor 51 and an image generated by the imaging device 41 with each other. In this case, the light emitted by the illumination light source 42 included in the insertion unit 4 can be entirely used as the light indicating the measurement operation. Thus, no design change of the main body of the endoscope is necessary. A user has only to attach the sensor unit 5c to the insertion unit 4, and the burden of the user can be reduced.

### Embodiment of Second Invention

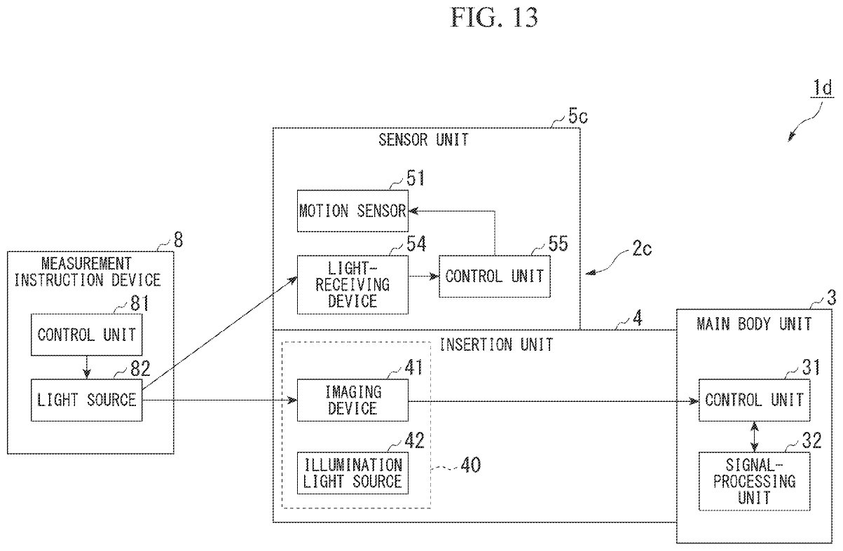

FIG. 13 shows a configuration of an endoscope system 1d according to an embodiment of a second invention related to the present invention. The same configuration as that shown in FIG. 1 or FIG. 12 will not be described.

The endoscope system 1d shown in FIG. 13 includes an endoscope 2c, a main body unit 3, and a measurement instruction device 8. The endoscope 2 shown in FIG. 1 is changed to the endoscope 2c. The endoscope 2c is the same as the endoscope 2c shown in FIG. 12. The measurement instruction device 8 includes a control unit 81 and a light source 82.

The control unit 81 instructs the light source 82 to generate light indicating a start of the measurement operation. Thereafter, the control unit 81 instructs the light source 82 to generate light indicating a stoppage of the measurement operation. The control unit 81 may be constituted by at least one of a processor and a logic circuit. The control unit 81 may include one or a plurality of processors. The control unit 81 may include one or a plurality of logic circuits.

For example, the light source 82 is an LED. The light source 82 generates light for notifying the sensor unit 5c to start or stop the measurement operation. When the control unit 81 instructs the light source 82 to generate the light indicating the start of the measurement operation, the light source 82 generates light having a pattern of the light-emission state corresponding to the start of the measurement operation. In other words, the light source 82 generates the light indicating the start of the measurement operation. Thereafter, when the control unit 81 instructs the light source 82 to generate the light indicating the stoppage of the measurement operation, the light source 82 generates light having a pattern of the light-emission state corresponding to the stoppage of the measurement operation. In other words, the light source 82 generates the light indicating the stoppage of the measurement operation.

The light generated by the light source 82 is incident on the light-receiving device 54 of the sensor unit 5c and the imaging device 41 of the insertion unit 4. The light-receiving device 54 receives the light generated by the light source 82 and generates a signal in accordance with the amount of the light. The signal is converted into a digital signal by an A/D converter not shown in FIG. 13 and is output to the control unit 55.

The control unit 55 detects the light indicating the start of the measurement operation or the stoppage of the measurement operation as with the embodiment of the first invention. The control unit 55 outputs a control signal indicating the start of the measurement operation or the stoppage of the measurement operation to the motion sensor 51 and controls the measurement operation executed by the motion sensor 51 as with the embodiment of the first invention. The motion sensor 51 starts or stops the measurement operation on the basis of the control signal output from the control unit 55 as with the embodiment of the first invention.

The imaging device 41 receives the light generated by the light source 82 and generates an image. The imaging device 41 outputs the generated image to the control unit 31 of the main body unit 3. The control unit 31 outputs the image output from the imaging device 41 to the signal-processing unit 32. The signal-processing unit 32 detects the light generated by the light source 82 by processing the image.

When the light indicating the start of the measurement operation is detected, the signal-processing unit 32 notifies the control unit 31 of the start of the measurement operation. The control unit 31 regards a time point at which the start of the measurement operation is reported as a time point at which the motion sensor 51 starts the measurement operation. In this way, the control unit 31 can determine an image generated at a timing at which the motion sensor 51 starts the measurement operation. When the light indicating the stoppage of the measurement operation is detected, the signal-processing unit 32 notifies the control unit 31 of the stoppage of the measurement operation. The control unit 31 regards a time point at which the stoppage of the measurement operation is reported as a time point at which the motion sensor 51 stops the measurement operation. In this way, the control unit 31 can determine an image generated at a timing at which the motion sensor 51 stops the measurement operation.

For example, the measurement instruction device 8 is disposed outside an object that is an observation target. Before the endoscope 2c is inserted into the object, a user puts the endoscope 2c close to the measurement instruction device 8 and inputs an instruction for starting measurement into the measurement instruction device 8. At this time, the control unit 81 instructs the light source 82 to generate the light indicating the start of the measurement operation. Thereafter, the endoscope 2c is inserted into the object.

A user pulls the endoscope 2c out of the object in order to stop measurement. After the endoscope 2c is pulled out of the object, a user puts the endoscope 2c close to the measurement instruction device 8 and inputs an instruction for stopping measurement into the measurement instruction device 8. At this time, the control unit 81 instructs the light source 82 to generate the light indicating the stoppage of the measurement operation.

The sensor unit 5c may include the communication unit 53 shown in FIG. 6, and the main body unit 3 may include the communication unit 33 shown in FIG. 6. In such a case, the communication unit 53 may transmit the measurement value output from the motion sensor 51 to the communication unit 33. The signal-processing unit 32 may detect the light generated by the light source 82 by processing the image. The signal-processing unit 32 may associate a time point at which the measurement operation is executed and a time point at which the image is generated with each other so as to associate the measurement value and the image with each other. The control unit 31 may record the measurement value and the image on the memory 34.

In a case in which the sensor unit 5c includes the communication unit 53 and the main body unit 3 includes the communication unit 33, the endoscope system 1d may include the external terminal 6 shown in FIG. 8. The communication unit 53 may transmit the measurement value output from the motion sensor 51 to the communication unit 61 of the external terminal 6. The communication unit 33 may transmit the image recorded on the memory 34 to the communication unit 61 of the external terminal 6. The signal-processing unit 63 of the external terminal 6 may detect the light generated by the light source 82 by processing the image. The signal-processing unit 63 may associate a time point at which the measurement operation is executed and a time point at which the image is generated with each other so as to associate the measurement value and the image with each other. The control unit 62 may record the measurement value and the image on the memory 64.

The sensor unit 5c may be disposed in an adaptor used for an endoscope.

In the embodiment of the second invention, the illumination light source 82 generates light indicating the measurement operation, and the control unit 55 controls the measurement operation executed by the motion sensor 51 on the basis of the light received by the light-receiving device 54. The motion sensor 51 executes the measurement operation and generates a measurement value in a period specified by the measurement instruction device 8. The signal-processing unit 32 detects the light indicating the measurement operation by processing the image. Therefore, the endoscope system 1d can synchronize a measurement value generated by the motion sensor 51 and an image generated by the imaging device 41 with each other.

### Embodiment of Third Invention

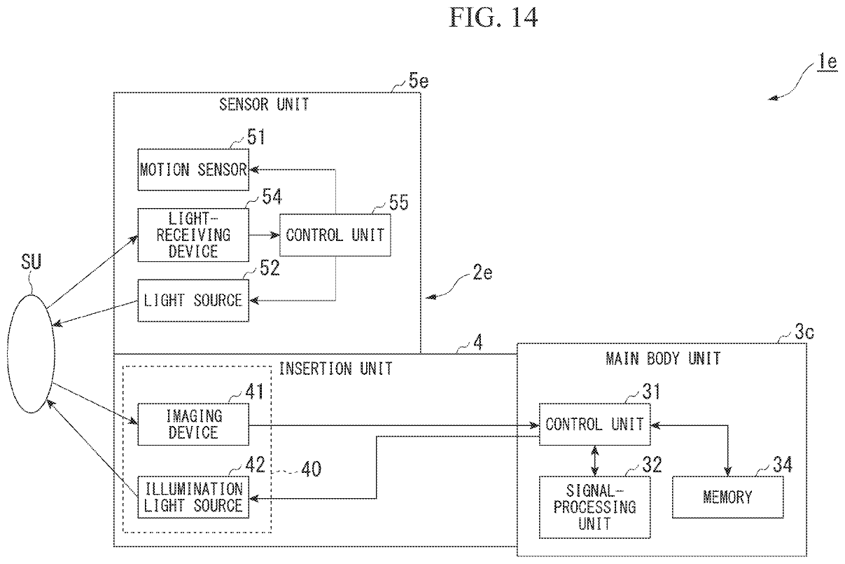

FIG. 14 shows a configuration of an endoscope system 1e according to an embodiment of a third invention related to the present invention. The same configuration as that shown in FIG. 1 or FIG. 12 will not be described.

The endoscope 2 shown in FIG. 1 is changed to an endoscope 2e. The main body unit 3 shown in FIG. 1 is changed to a main body unit 3c. The main body unit 3c is the same as the main body unit 3c shown in FIG. 12. The sensor unit 5 shown in FIG. 1 is changed to a sensor unit 5e. The sensor unit 5e includes a motion sensor 51, a light source 52, a light-receiving device 54, and a control unit 55.

The control unit 31 of the main body unit 3c instructs the illumination light source 42 to generate light indicating a start of the measurement operation at a timing at which recording of an image is started as with the embodiment of the first invention. The control unit 31 instructs the illumination light source 42 to generate light indicating a stoppage of the measurement operation at a timing at which the recording of an image is stopped as with the embodiment of the first invention. After the recording of an image is stopped, the control unit 31 instructs the illumination light source 42 to generate light indicating transmission of a measurement value in order to acquire the measurement value.

The illumination light source 42 of the insertion unit 4 generates light for requesting the sensor unit 5e to start the measurement operation, stop the measurement operation, or transmit the measurement value. The illumination light source 42 generates the light indicating the start of the measurement operation at a timing at which the recording of an image is started as with the embodiment of the first invention. The illumination light source 42 generates the light indicating the stoppage of the measurement operation at a timing at which the recording of an image is stopped as with the embodiment of the first invention. Thereafter, when the control unit 31 instructs the illumination light source 42 to generate the light indicating the transmission of a measurement value, the illumination light source 42 generates light having a pattern of the light-emission state corresponding to the transmission of a measurement value. In other words, the illumination light source 42 generates the light indicating the transmission of a measurement value.

The light-receiving device 54 receives the light generated by the illumination light source 42 and generates a signal in accordance with the amount of the light as with the embodiment of the first invention. The signal is converted into a digital signal by an A/D converter not shown in FIG. 14 and is output to the control unit 55.

The control unit 55 analyzes a pattern of the light-emission state on the basis of the intensity of light indicated by the value of the digital signal as with the embodiment of the first invention. In this way, the control unit 55 detects the light indicating any one of the start of the measurement operation, the stoppage of the measurement operation, and the transmission of a measurement value. When the light indicating the start of the measurement operation is detected, the control unit 55 outputs a control signal indicating the start of the measurement operation to the motion sensor 51 and causes the motion sensor 51 to start the measurement operation. When the light indicating the stoppage of the measurement operation is detected, the control unit 55 outputs a control signal indicating the stoppage of the measurement operation to the motion sensor 51 and causes the motion sensor 51 to stop the measurement operation. When the light indicating the transmission of a measurement value is detected, the control unit 55 instructs the light source 52 to generate light indicating a measurement value. The light source 52 generates light having a pattern of the light-emission state corresponding to a measurement value. In other words, the light source 52 generates light indicating a measurement value.

The imaging device 41 receives the light generated by the light source 52 and generates an image. The imaging device 41 outputs the generated image to the control unit 31 of the main body unit 3c. The control unit 31 outputs the image output from the imaging device 41 to the signal-processing unit 32. The signal-processing unit 32 detects a measurement value by processing the image. The signal-processing unit 32 notifies the control unit 31 of the detected measurement value. The control unit 31 associates the measurement value with the image stored on the memory 34 and stores the measurement value on the memory 34.

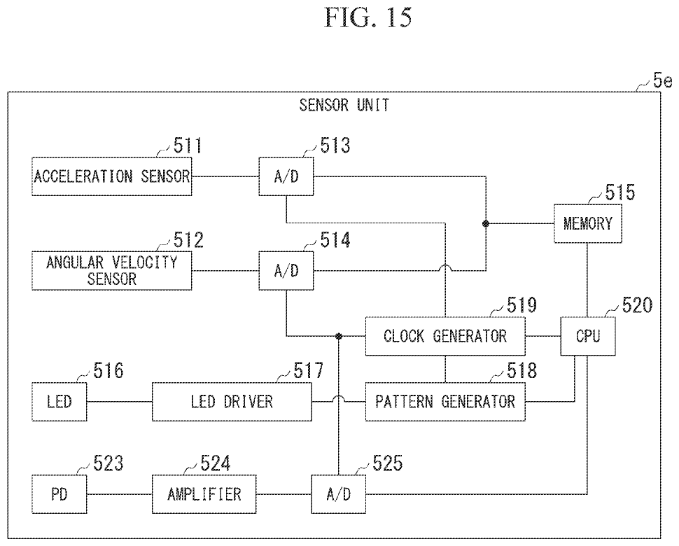

FIG. 15 shows a configuration of the sensor unit 5e. The sensor unit 5e includes an acceleration sensor 511, an angular velocity sensor 512, an A/D converter 513, an A/D converter 514, a memory 515, an LED 516, an LED driver 517, a pattern generator 518, a clock generator 519, a CPU 520, a PD 523, an amplifier 524, and an A/D converter 525. The same configuration as that shown in FIG. 11 will not be described.

The LED 516 corresponds to the light source 52 shown in FIG. 14. The CPU 520 corresponds to the control unit 55 shown in FIG. 14. The PD 523 is a photodiode and corresponds to the light-receiving device 54 shown in FIG. 14. The PD 523 receives the light generated by the illumination light source 42 and generates an analog signal in accordance with the amount of the light. The amplifier 524 amplifies the analog signal output from the PD 523. The A/D converter 525 performs AD conversion on the analog signal output from the amplifier 524 and converts the analog signal into a digital signal. The A/D converter 525 outputs the digital signal to the CPU 520.

The CPU 520 outputs a measurement value stored on the memory 515 to the pattern generator 518. The pattern generator 518 generates a pattern of the light-emission state corresponding to the measurement value. The pattern generator 518 outputs a control signal corresponding to the pattern to the LED driver 517. The LED driver 517 controls the operation of the LED 516 on the basis of the control signal. The LED 516 generates light indicating the measurement value.

The sensor unit 5e may be disposed in an adaptor used for an endoscope.

In the embodiment of the third invention, the illumination light source 42 generates light indicating the measurement operation, and the control unit 55 controls the measurement operation executed by the motion sensor 51 on the basis of the light received by the light-receiving device 54. The motion sensor 51 executes the measurement operation and generates a measurement value in a period during which an image is recorded on the memory 34. Therefore, the endoscope system 1e can synchronize a measurement value generated by the motion sensor 51 and an image generated by the imaging device 41 with each other.

The light source 52 generates light indicating a measurement value and the imaging device 41 generates an image in which the light is seen. The signal-processing unit 32 detects a measurement value by processing the image. Therefore, the sensor unit 5e does not need to include the communication unit 53 shown in FIG. 6, and the main body unit 3c does not need to include the communication unit 33 shown in FIG. 6. In this case, since the illumination light source 42 generates light indicating the measurement operation and the light source 52 generates light indicating a measurement value, the sensor unit 5e can directly transmit the measurement value of the motion sensor 51 to the main body unit 3c without executing analysis processing.

### Embodiment of Fourth Invention

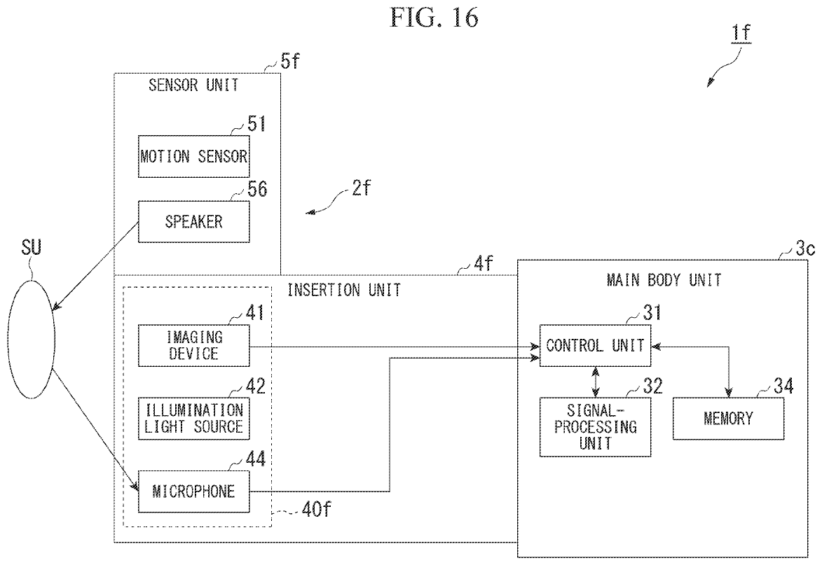

FIG. 16 shows a configuration of an endoscope system if according to an embodiment of a fourth invention related to the present invention. The same configuration as that shown in FIG. 1 or FIG. 12 will not be described.

The endoscope 2 shown in FIG. 1 is changed to an endoscope 2f. The main body unit 3 shown in FIG. 1 is changed to a main body unit 3c. The main body unit 3c is the same as the main body unit 3c shown in FIG. 12. The insertion unit 4 shown in FIG. 1 is changed to an insertion unit 4f. The insertion unit 4f includes a microphone 44 in addition to the imaging device 41 and the illumination light source 42 shown in FIG. 1. The sensor unit 5 shown in FIG. 1 is changed to a sensor unit 5f. The sensor unit 5f includes a motion sensor 51 and a speaker 56.

The speaker 56 generates a sound indicating a timing at which the measurement operation is executed. The speaker 56 generates a sound having a pattern corresponding to a start of the measurement operation or a stoppage of the measurement operation. The speaker 56 generates a sound indicating the start of the measurement operation at which the motion sensor 51 starts the measurement operation. The speaker 56 generates a sound indicating the stoppage of the measurement operation at which the motion sensor 51 stops the measurement operation.

The sound generated by the speaker 56 reaches the microphone 44. The microphone 44 is disposed in a distal end part 40f of the insertion unit 4f. The microphone 44 receives the sound generated by the speaker 56 and generates a signal in accordance with the amount of the sound. The signal is converted into a digital signal by an A/D converter not shown in FIG. 16 and is output to the control unit 31. The microphone 44 may be disposed in the main body unit 3c.

The control unit 31 outputs the digital signal to the signal-processing unit 32. The signal-processing unit 32 analyzes a pattern of a sound on the basis of the intensity of the sound indicated by the digital signal. In this way, the signal-processing unit 32 detects the sound indicating the start of the measurement operation or the stoppage of the measurement operation.

When the sound indicating the start of the measurement operation is detected, the signal-processing unit 32 notifies the control unit 31 of the start of the measurement operation. The control unit 31 regards a time point at which the start of the measurement operation is reported as a time point at which the motion sensor 51 starts the measurement operation. In this way, the control unit 31 can determine an image generated at a timing at which the motion sensor 51 starts the measurement operation. When the sound indicating the stoppage of the measurement operation is detected, the signal-processing unit 32 notifies the control unit 31 of the stoppage of the measurement operation. The control unit 31 regards a time point at which the stoppage of the measurement operation is reported as a time point at which the motion sensor 51 stops the measurement operation. In this way, the control unit 31 can determine an image generated at a timing at which the motion sensor 51 stops the measurement operation.

The sensor unit 5f may include the communication unit 53 shown in FIG. 6, and the main body unit 3c may include the communication unit 33 shown in FIG. 6. In such a case, the communication unit 53 may transmit the measurement value output from the motion sensor 51 to the communication unit 33. The control unit 31 may associate the measurement value with the image recorded on the memory 34 and may record the measurement value on the memory 34.

The sensor unit 5f may be disposed in an adaptor used for an endoscope.

In the embodiment of the fourth invention, the speaker 56 generates a sound indicating the measurement operation, and the signal-processing unit 32 detects the measurement operation on the basis of the sound detected by the microphone 44. Therefore, the endoscope system if can synchronize a measurement value generated by the motion sensor 51 and an image generated by the imaging device 41 with each other.

### Embodiment of Fifth Invention

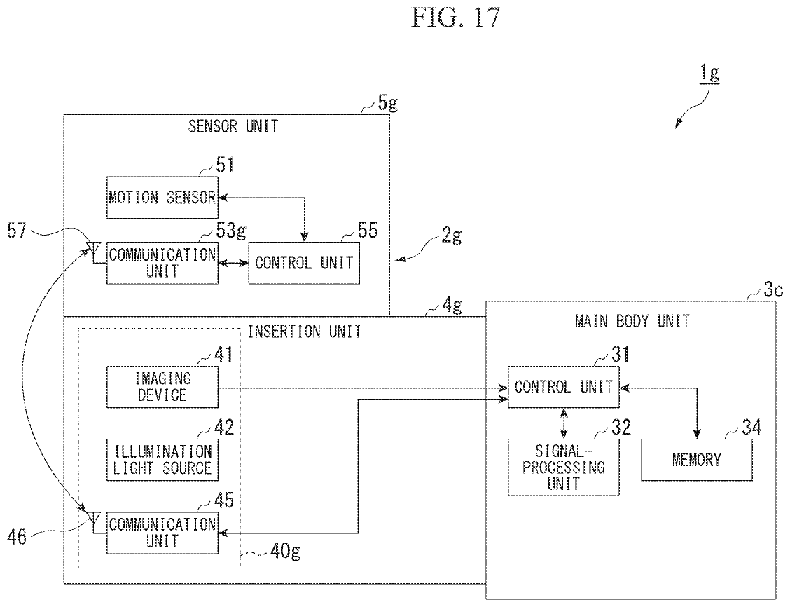

FIG. 17 shows a configuration of an endoscope system 1g according to an embodiment of a fifth invention related to the present invention. The same configuration as that shown in FIG. 1 or FIG. 12 will not be described.

The endoscope 2 shown in FIG. 1 is changed to an endoscope 2g. The main body unit 3 shown in FIG. 1 is changed to a main body unit 3c. The main body unit 3c is the same as the main body unit 3c shown in FIG. 12. The insertion unit 4 shown in FIG. 1 is changed to an insertion unit 4g. The insertion unit 4g includes a communication unit 45 and an antenna 46 in addition to the imaging device 41 and the illumination light source 42 shown in FIG. 1. The sensor unit 5 shown in FIG. 1 is changed to a sensor unit 5g. The sensor unit 5g includes a motion sensor 51, a communication unit 53g, a control unit 55, and an antenna 57.

The communication unit 53g includes a wireless communication circuit and is connected to the antenna 57. The communication unit 53g performs wireless communication with the communication unit 45 of the insertion unit 4g via the antenna 57. The antenna 57 performs transmission and reception of radio waves. The control unit 55 is the same as the control unit 55 shown in FIG. 12.

The communication unit 45 and the antenna 46 are disposed in a distal end part 40g of the insertion unit 4g. The communication unit 45 includes a wireless communication circuit and is connected to the antenna 46. The communication unit 45 performs wireless communication with the communication unit 53g of the sensor unit 5g via the antenna 46. The antenna 46 performs transmission and reception of radio waves. The communication unit 45 and the antenna 46 may be disposed in the main body unit 3c.

The control unit 31 of the main body unit 3c instructs the communication unit 45 to transmit start information indicating a start of the measurement operation at a timing at which recording of an image is started. The communication unit 45 transmits the start information to the communication unit 53g of the sensor unit 5g by using the antenna 46. The antenna 46 transmits radio waves corresponding to the start information.

The antenna 57 of the sensor unit 5g receives the radio waves corresponding to the start information transmitted by the communication unit 45 of the insertion unit 4g. The communication unit 53g receives the start information from the communication unit 45 via the antenna 57. The communication unit 53g outputs the received start information to the control unit 55. When the start information is received, the control unit 55 outputs a control signal indicating a start of the measurement operation to the motion sensor 51 and causes the motion sensor 51 to start the measurement operation.

The control unit 31 of the main body unit 3c instructs the communication unit 45 to transmit stop information indicating a stoppage of the measurement operation at a timing at which recording of an image is stopped. The communication unit 45 transmits the stop information to the communication unit 53g of the sensor unit 5g by using the antenna 46. The antenna 46 transmits radio waves corresponding to the stop information.

The antenna 57 of the sensor unit 5g receives the radio waves corresponding to the stop information transmitted by the communication unit 45 of the insertion unit 4g. The communication unit 53g receives the stop information from the communication unit 45 via the antenna 57. The communication unit 53g outputs the received stop information to the control unit 55. When the stop information is received, the control unit 55 outputs a control signal indicating a stoppage of the measurement operation to the motion sensor 51 and causes the motion sensor 51 to stop the measurement operation.

The motion sensor 51 outputs a measurement value to the control unit 55. The control unit 55 instructs the communication unit 53g to transmit the measurement value. The communication unit 53g transmits the measurement value to the communication unit 45 of the insertion unit 4g via the antenna 57. The antenna 57 transmits radio waves corresponding to the measurement value.

The antenna 46 of the insertion unit 4g receives the radio waves corresponding to the measurement value transmitted by the communication unit 53g of the sensor unit 5g. The communication unit 45 receives the measurement value from the communication unit 53g via the antenna 46. The communication unit 45 outputs the received measurement value to the control unit 31. The control unit 31 associates the measurement value with the image stored on the memory 34 and stores the measurement value on the memory 34.

The sensor unit 5g may be disposed in an adaptor used for an endoscope.

In the embodiment of the fifth invention, the communication unit 45 transmits information indicating the measurement operation to the communication unit 53g, and the control unit 55 controls the measurement operation executed by the motion sensor 51 on the basis of the information received by the communication unit 53g. The motion sensor 51 executes the measurement operation and generates a measurement value in a period during which an image is recorded on the memory 34. Therefore, the endoscope system 1g can synchronize a measurement value generated by the motion sensor 51 and an image generated by the imaging device 41 with each other.

### Embodiment of Sixth Invention

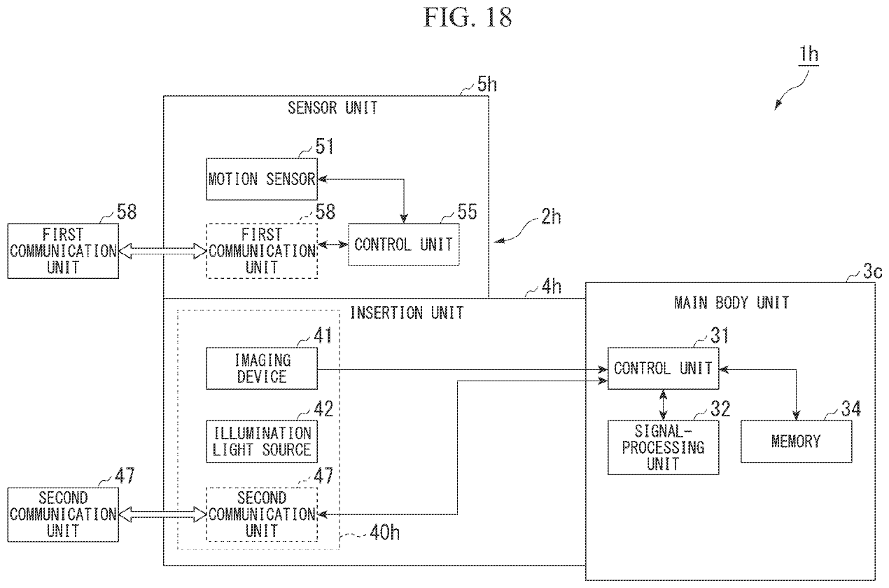

FIG. 18 shows a configuration of an endoscope system 1h according to an embodiment of a sixth invention related to the present invention. The same configuration as that shown in FIG. 1 or FIG. 12 will not be described.

The endoscope 2 shown in FIG. 1 is changed to an endoscope 2h. The main body unit 3 shown in FIG. 1 is changed to a main body unit 3c. The main body unit 3c is the same as the main body unit 3c shown in FIG. 12. The insertion unit 4 shown in FIG. 1 is changed to an insertion unit 4h. The insertion unit 4h includes a second communication unit 47 in addition to the imaging device 41 and the illumination light source 42 shown in FIG. 1. The sensor unit 5 shown in FIG. 1 is changed to a sensor unit 5h. The sensor unit 5h includes a motion sensor 51, a control unit 55, and a first communication unit 58.

The first communication unit 58 is attachable to and detachable from the sensor unit 5h. The first communication unit 58 is connected to the sensor unit 5h when the first communication unit 58 is connected to a connecter provided in the sensor unit 5h.

The second communication unit 47 is attachable to and detachable from the insertion unit 4h. The second communication unit 47 is connected to the insertion unit 4h when the second communication unit 47 is connected to a connecter provided in a distal end part 40h of the insertion unit 4h.

Two or more types of the first communication unit 58 and two or more types of the second communication unit 47 are prepared. By using the first communication unit 58 without using the second communication unit 47, any one of the first to third embodiments of the present invention is realized. In addition, by using the first communication unit 58 without using the second communication unit 47 or by combining the first communication unit 58 and the second communication unit 47, any one of the embodiments of the first to fifth inventions related to the present invention is realized.

For example, the first communication unit 58 includes the light source 52, and the second communication unit 47 is not used. In this case, any one of the first to third embodiments (FIG. 1, FIG. 6, and FIG. 8) of the present invention is realized.

For example, the first communication unit 58 includes the light-receiving device 54, and the second communication unit 47 is not used. In this case, any one of the embodiments (FIG. 12 and FIG. 13) of the first and second inventions related to the present invention is realized. In the embodiment of the second invention related to the present invention, the measurement instruction device 8 is necessary.

For example, the first communication unit 58 includes the light source 52 and the light-receiving device 54, and the second communication unit 47 is not used. In this case, the embodiment (FIG. 14) of the third invention related to the present invention is realized.

For example, the first communication unit 58 includes the speaker 56, and the second communication unit 47 includes the microphone 44. In this case, the embodiment (FIG. 16) of the fourth invention related to the present invention is realized.

For example, the first communication unit 58 includes the communication unit 53g and the antenna 57, and the second communication unit 47 includes the communication unit 45 and the antenna 46. In this case, the embodiment (FIG. 17) of the fifth invention related to the present invention is realized.

The microphone 44 may be built in the insertion unit 4h or the main body unit 3c. Alternatively, the communication unit 45 and the antenna 46 may be built in the insertion unit 4h or the main body unit 3c. In a case in which the microphone 44 is built in the insertion unit 4h or the main body unit 3c, the second communication unit 47 does not need to include the microphone 44. In a case in which the communication unit 45 and the antenna 46 are built in the insertion unit 4h or the main body unit 3c, the second communication unit 47 does not need to include the communication unit 45 and the antenna 46. In a case in which the microphone 44, the communication unit 45, and the antenna 46 are built in the insertion unit 4h or the main body unit 3c, the second communication unit 47 is unnecessary.

The sensor unit 5h may be disposed in an adaptor used for an endoscope. In this way, the fourth embodiment (FIG. 10 and FIG. 11) of the present invention is realized.

In the embodiment of the sixth invention, the endoscope system 1h can realize any one of the first to third embodiments of the present invention or any one of the embodiments of the first to fifth inventions related to the present invention.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An endoscope system, comprising an endoscope comprising:
an insertion unit; and
a sensor disposed at a distal end of the insertion unit, the sensor comprising:
a motion sensor configured to execute a measurement operation and generate a measurement value indicating a measurement result; and
a light source configured to generate light indicating the measurement operation at a timing that is synchronized with the measurement operation,
wherein the insertion unit comprises an imaging device disposed at the distal end and configured to receive the light generated by the light source and generate an image.

2. The endoscope system according to claim 1, further comprising a processor configured to detect the light generated by the light source by processing the image and associate a time point at which the measurement operation is executed and a time point at which the image is generated with each other so as to associate the measurement value and the image with each other.

3. The endoscope system according to claim 2,
wherein the light source is configured to generate the light at a first timing at which the motion sensor starts the measurement operation, and
the processor is configured to detect the light generated at the first timing by the light source.

4. The endoscope system according to claim 3,
wherein the light source is configured to generate the light at a second timing at which the motion sensor stops the measurement operation, and
the processor is configured to detect the light generated at the second timing by the light source.

5. The endoscope system according to claim 3,
wherein the motion sensor comprises:
an acceleration sensor configured to execute the measurement operation; and
an angular velocity sensor configured to execute the measurement operation.

6. The endoscope system according to claim 3,
wherein the light source is configured to generate light indicating that the measurement operation continues while the motion sensor executes the measurement operation, and
the processor is configured to detect the light indicating that the measurement operation continues by processing the image.

7. The endoscope system according to claim 3, further comprising a memory configured to store the measurement value and the image associated with each other.

8. The endoscope system according to claim 1,
wherein the light source is capable of switching between a first state in which the light source generates light and a second state in which the light source stops light-emission, and
a pattern in which the first state occurs corresponds to a state of the measurement operation.

9. The endoscope system according to claim 8,
wherein a number of periods during which the first state occurs corresponds to the state of the measurement operation.

10. The endoscope system according to claim 8,
wherein a length of a period during which the first state continues corresponds to the state of the measurement operation.

11. The endoscope system according to claim 1,
wherein the sensor is attachable to and detachable from the distal end.

12. The endoscope system according to claim 11,
wherein the sensor is disposed in an optical adaptor for observation using the endoscope.

13. The endoscope system according to claim 1,
wherein the light source is configured to generate visible light.

14. The endoscope system according to claim 1,
wherein the light source is configured to generate one of infrared light and ultraviolet light.

15. The endoscope system according to claim 1,
wherein the light source is configured to generate light having a wavelength corresponding to a state of the measurement operation.

16. An adaptor used for an endoscope and connected to a distal end of an insertion unit included in the endoscope, the adaptor comprising:
a motion sensor configured to execute a measurement operation and generate a measurement value indicating a measurement result;
a signal output circuit configured to output a control signal indicating a timing of the measurement operation to the motion sensor; and
a light source configured to generate light indicating the measurement operation at the timing indicated by the control signal.

17. A method of operating an endoscope, the method comprising:
a measurement step of causing a motion sensor disposed at a distal end of the endoscope to execute a measurement operation and generate a measurement value indicating a measurement result;
a light-emission step of causing a light source disposed at the distal end to generate light indicating the measurement operation at a timing that is synchronized with the measurement operation; and
an imaging step of causing an imaging device disposed at the distal end to receive the light generated by the light source and generate an image.
